(19) United States
(12) Patent Application Publication
(10) Pub. No.: US 2005/0245798 A1
(43) Pub. Date: Nov. 3, 2005

(54) Device and system for humor component detection

(75) Inventors: Masaki Yamaguchi, Toyama-city (JP); Kenichi Yanai, Nisshin-city (JP); Tadayoshi Taniyama, Yokohama-city (JP)

Correspondence Address: POSZ LAW GROUP, PLC, 12040 SOUTH LAKES DRIVE, SUITE 101, RESTON, VA, 20191, US

(73) Assignee: DENSO CORPORATION

(21) Appl. No.: 11/036,092

(22) Filed: Jan. 18, 2005

(30) Foreign Application Priority Data
Jan. 21, 2004 (JP) 2004-13372
Oct. 12, 2004 (JP) 2004-298163

(51) Int. Cl. A61B 5/00; C12Q 1/00; A61B 5/05; A61B 3/16

(52) U.S. Cl. 600/345; 128/903; 600/309; 435/4

(57) ABSTRACT

A device and a system for humor component detection are capable of continuously carrying out measurement with accuracy for a long time and are excellent from the aspect of good hygiene. The humor component detection system comprises the humor component detection device and an extra-corporeal sensing device. The humor component detection device comprises a biosensor, a microbe containing portion, and an outer shell. Here, the biosensor comprises a sensing element and an electronic device. The microbe containing portion includes a pouched microtube blanketing film into which the electrodes of the sensing element are inserted, a plurality of acinous polymer-filmed porous microcapsules contained inside the film, and GOD genetically-modified bacteria contained in the microcapsules.

### CROSS REFERENCE TO RELATED APPLICATIONS

This application is based on and incorporates herein by reference Japanese Patent Applications No. 2004-13372 filed on Jan. 21, 2004 and No. 2004-298163 filed on Oct. 12, 2004.

### FIELD OF THE INVENTION

The present invention relates to a device for humor component detection which is retained in a human body and is capable of continuously measuring humor components, and a system for humor component detection.

### BACKGROUND OF THE INVENTION

To mitigate complications of diabetes, a blood sugar level must be kept under control. However, patients with serious diabetes cannot control the blood sugar level by themselves. Therefore, an invasive blood sugar level measuring instrument is used to frequently collect blood from a finger and measure the blood sugar level. Then, based on the measured value, treatment is given to control the blood sugar.

However, with a method of measuring blood collected from a finger, it is difficult to continuously carry out measurement. Consequently, blood sugar level meters capable of continuous measurement are demanded.

As one of blood sugar level meters capable of continuous measurement, meters of such a type that a biosensor unit provided with a puncturing needle is buried in the human body for measurement are known (MINMED from Medtronic). In addition, a technique in which light of multiple wavelengths is applied from outside the body, and the resulting reflection spectrum is analyzed to continuously measure the blood sugar level is known. (Refer to Patent Document 1 and Patent Document 2.)
Patent Document 1: JP-H9-182739 A
Patent Document 2: U.S. Pat. No. 5,743,262 A (WO96/41151)

However, sensors of such a type that a biosensor unit is buried in the human body for measurement involve several problems. Enzymes required for the biosensor (enzymes indispensable to the process of chemical reaction in blood sugar level detection) are devitalized in a short time; therefore, the blood sugar level can be continuously measured only for three days or so. Protein sticks to the sensing portions of biosensors; therefore, the detecting capability of the biosensors is degraded. Retention of a puncturing needle in the body can lead to infectious diseases.

The method of analyzing the reflection spectrum involves the following problem: the absorption wavelengths of the components of internal blood sugar level do not have a characteristic waveform against the absorption waveform of the other components of blood. Therefore, with respect to this method, it is difficult to attain the accuracy at the same level as with invasive blood sugar level measuring instruments.

### SUMMARY OF THE INVENTION

The present invention has been made with the above problems taken into account. It is an object of the present invention to provide a device and a system for humor component detection which are capable of continuously carrying out measurement with accuracy for a long time and are excellent from the aspect of good hygiene.

(1) A humor component detection device according to a first aspect of the present invention is capable of continuously producing substances (e.g. enzymes) for use in a biosensor from organisms. For this reason, substances required for measurement with the biosensor is prevented from being devitalized, so that the detection device is capable of continuously carrying out measurement for a long time. The humor component detection device of the present invention can be retained in a living body (living organism), and is thus capable of continuous measurement.

One example of the above biosensor is one so designed that humor components are caused to electrochemically react by enzymes produced by organisms and the humor components are detected as electrical signals. The biosensor may be so constituted that it detects light produced by the reaction of humor components.

Examples of the above organisms include microbes, cells of human origin, tissues composed of the cells, mixtures of the cells and the tissues, and the like. For microbes, anaerobic ones are preferable.

There is no special limitation on the place in the living body in which the humor component detection device is retained as long as the humor exists there. Examples of such places include hypoderm, inside the mouth, the back of an eyelid, and the like.

(2) A humor component detection device in another aspect of the present invention uses microbes for the organism.

(3) A humor component detection device according to another aspect of the present invention uses genetically modified bacteria for the microbe. Therefore, the detection device can be used as a device for measuring the state of various diseases (e.g. lifestyle-related diseases, diabetes, hyperlipemia, pancreatitis) or a metabolism monitor. More specific description will be given. Genetically modified bacteria can be caused to produce various substances, such as enzymes, for use in measurement with the biosensor by genetic engineering. Therefore, genetically modified bacteria can be caused to produce a substance required for measurement of items corresponding to a specific disease, and thereby the detection device can be used as a device for carrying out measurement related to that disease.

(4) In a humor component detection device according to another aspect of the present invention, the organism is any of (A) cells having a genetic state for autoreproduction, (B) tissues composed of the cells, and (C) a mixture of the cells and the tissues.

According to the present invention, the following can be used for the organism: cells in which a virus has been genetically introduced so as to produce a substance (e.g. GOD) for use in a biosensor; tissues composed of the cells; and a mixture of the cells and the tissues.

(5) In a humor component detection device according to another aspect of the present invention, cells as the organism are of human origin. For this reason, if cells, tissues, or a mixture of them should leak from the humor component detection device, they have no harm to the subject. There is no possibility of defect in cells themselves or diffusion of toxic substances into the living body; therefore, accurate measurement can be carried out for a long time.

(6) In a humor component detection device according to another aspect of the present invention, the organism is covered with an organism diffusion preventing film which inhibits the permeation of the organism. Therefore, there is no possibility that the organism leaks to the outside.

An example of the organism diffusion preventing film is a film whose pore size or mesh size is smaller than the organism. For the organism diffusion preventing film, a wide variety of materials, such as polymeric materials used in medical care and other fields, can be used. Specific examples of the materials include: silicone, polyvinyl chloride, polymethylmethacrylate, polytetrafluoroethylene, polyester, polypropylene, polyurethane, cellulose, polystyrene, nylon, polycarbonate, polysulfone, polyacrylonitrile, polyvinyl alcohol, and the like.

(7) In a humor component detection device according to another aspect of the present invention, a detecting element which detects the humor components in a biosensor is covered with an interfering substance preventing film which limits the permeation of interfering substances (e.g. various proteins and the like existing in the humor) against detection. Therefore, the interfering substances are prevented from sticking to the detecting element, and accurate measurement can be constantly carried out. The interfering substance preventing film permits the permeation of humor components as the objects of measurement, and does not interfere with measurement.

An example of the interfering substance preventing film is a film whose pore size or mesh size is smaller than interfering substances and larger than the humor components as the objects of measurement. For the interfering substance preventing film, a wide variety of materials, such as polymeric materials used in medical care and other fields, can be used. Specific examples of the materials include: silicone, polyvinyl chloride, polymethyl methacrylate, polytetrafluoroethylene, polyester, polypropylene, polyurethane, cellulose, polystyrene, nylon, polycarbonate, polysulfone, polyacrylonitrile, polyvinyl alcohol, and the like.

(8) In a humor component detection device according to another aspect of the present invention, a detecting element which detects the humor components in a biosensor is covered with a produced substance diffusion preventing film which limits the permeation of substances produced by the organism. The organism is contained inside the produced substance diffusion preventing film. For this reason, the substances produced by the organism stay inside the produced substance diffusion preventing film. The concentration of the substances produced by the organism is sufficiently enhanced in proximity to the detecting element existing inside the produced substance diffusion preventing film. As a result, the humor component detection device of the present invention is capable of accurately carrying out measurement. The produced substance diffusion preventing film permits the permeation of humor components as the objects of measurement, and does not interfere with measurement.

An example of the produced substance diffusion preventing film is a film whose pore size or mesh size is smaller than the substances produced by the organism and larger than humor components as the objects of measurement. For the produced substance diffusion preventing film, a wide variety of materials, such as polymeric materials used in medical care and other fields, can be used. Specific examples of the materials include: silicone, polyvinyl chloride, polymethyl methacrylate, polytetrafluoroethylene, polyester, polypropylene, polyurethane, cellulose, polystyrene, nylon, polycarbonate, polysulfone, polyacrylonitrile, polyvinyl alcohol, and the like.

(9) A humor component detection device according to another aspect of the present invention is provided with an outer shell, and can be hypodermically implanted with ease. The outer shell permits the permeation of humor components as the objects of measurement, and does not interfere with measurement.

An example of the outer shell is a film whose pore size or mesh size is larger than humor components as the objects of measurement. For the outer shell, a wide variety of materials, such as polymeric materials used in medical care and other fields, can be used. Specific examples of the materials include: silicone, polyvinyl chloride, polymethyl methacrylate, polytetrafluoroethylene, polyester, polypropylene, polyurethane, cellulose, polystyrene, nylon, polycarbonate, polysulfone, polyacrylonitrile, polyvinyl alcohol, and the like.

(10) A humor component detection device according to another aspect of the present invention is provided with electrodes as a detecting element which electrodes are capable of detecting electrical signals produced when the humor components undergo electrochemical reaction. For this reason, the humor components can be measured with accuracy.

(11) In a humor component detection device according to another aspect of the present invention, electrodes are chemically modified with a living body functional polymer. For this reason, the interfering substances in the humor can be more effectively prevented from sticking to the electrodes, and accurate measurement can be carried out for a long time.

For the living body functional polymer, a wide variety of polymeric materials, such as those used in medical care and other fields, can be used. Specific examples of the materials include: silicone, polyvinyl chloride, polymethyl methacrylate, polytetrafluoroethylene, polyester, polypropylene, polyurethane, cellulose, polystyrene, nylon, polycarbonate, polysulfone, polyacrylonitrile, polyvinyl alcohol, and the like.

(12) A humor component detection device according to another aspect of the present invention is capable of transmitting data obtained as the result of detection by a biosensor to the outside by a transmitting unit.

(13) A humor component detection device according to another aspect of the present invention is capable of wirelessly transmitting data by a transmitting unit in real time.

(14) In a humor component detection system according to another aspect of the present invention, data obtained as the result of measurement with a humor component detection device can be monitored by an extra-corporeal monitoring device.

(15) In a humor component detection system according to another aspect of the present invention, received data can be recorded together with time data by a data recording unit. For this reason, how the concentration of humor components varies with a lapse of time can be monitored for a long time.

(16) In a humor component detection system according to another aspect of the present invention, whether received data is abnormal or not can be determined by a determining unit provided in an extra-corporeal monitoring device. When data is determined to be abnormal, that can be notified of by a notifying unit. Thus, the user can swiftly learn any anomaly in measurement data.

### BRIEF DESCRIPTION OF THE DRAWINGS

The above and other objects, features, and advantages of the present invention will become more apparent from the following detailed description made with reference to the accompanying drawings. In the drawings:

FIG. 1 is an explanatory drawing illustrating the configuration of a humor component detection system;

FIG. 2 is an explanatory drawing illustrating the configuration of a humor component detection device;

FIG. 3 is a block diagram illustrating the electrical constitution of a humor component detection system;

FIG. 4 is an explanatory drawing illustrating the configuration of a humor component detection device;

FIG. 5 is an explanatory drawing illustrating electrochemical reactions which occur in an humor component detection device;

FIG. 6 is an explanatory drawing illustrating the configuration of a humor component detection device;

FIG. 7 is an explanatory drawing illustrating a method for creating modified cells; and

FIG. 8 is a sequence listing showing a base sequence of the X56443 gene.

### DETAILED DESCRIPTION OF THE PREFERRED EMBODIMENTS

Hereafter, description will be given to modes (embodiments) in which a humor component detection device and a humor component detection system of the present invention are realized. Here, a humor component detection device and a humor component detection system whose object of measurement is blood sugar (glucose), one of the humor components, will be taken as an example.

### First Embodiment

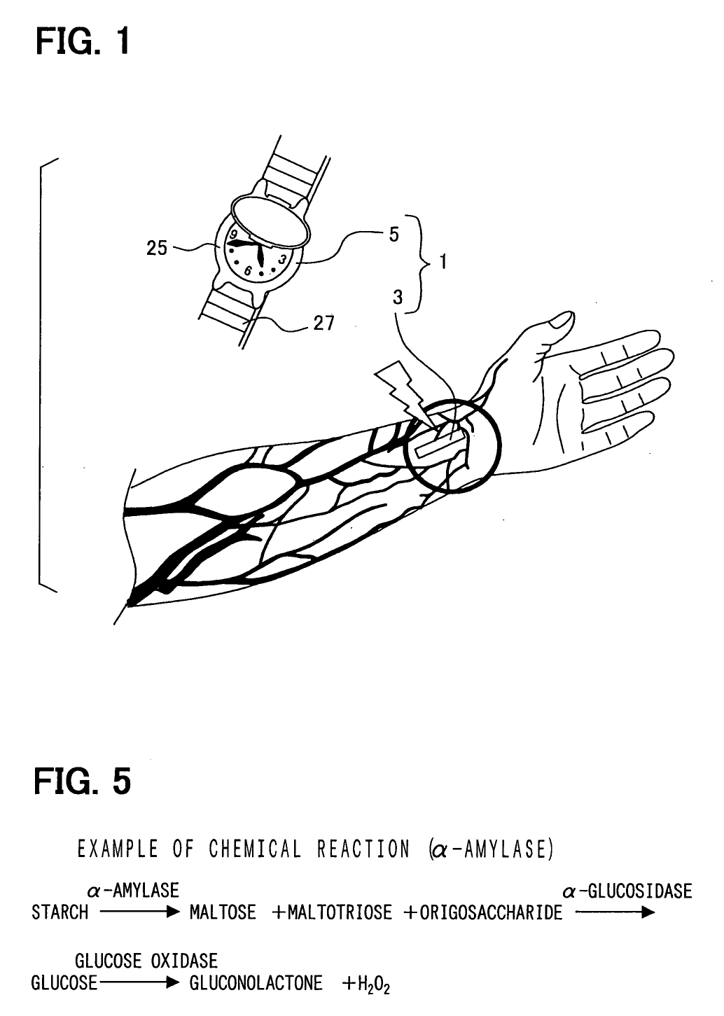

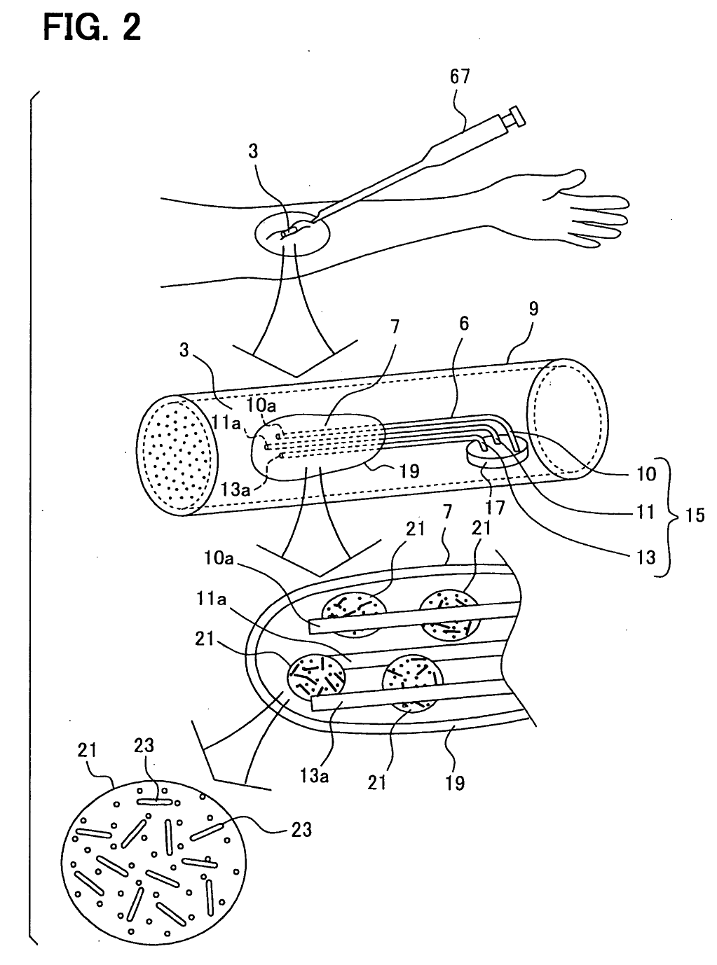

(a) First, general description will be given to the configuration of a humor component detection system of first embodiment with reference to FIG. 1 and FIG. 2. As illustrated in FIG. 1, the humor component detection system 1 comprises: a humor component detection device 3 implanted in a living body (or living organism) (hypodermically); and an extra-corporeal sensing device (extra-corporeal monitoring device) 5 which can be worn around a wrist like a wrist watch.

As illustrated in FIG. 2, the humor component detection device 3 comprises a biosensor 6, a microbe containing portion 7, and an outer shell 9. The biosensor 6 has a sensing element (detecting element) 15 comprising three electrodes: a working electrode 10 made of platinum, a counter electrode 11 made of platinum, and a reference electrode 13 made of silver. The biosensor 6 is further provided with an electronic device 17 connected to the sensing element 15.

As illustrated in FIG. 2, the microbe containing portion 7 comprises: a pouched microtube blanketing film 19 into which the tip 10a of the working electrode 10, the tip 11a of the counter electrode 11, and the tip 13a of the reference electrode 13 are inserted; a plurality of acinous polymer-filmed porous microcapsules 21 contained inside the microtube blanketing film 19; and GOD genetically-modified bacteria (transgenics bacteria) 23 contained inside the polymer-filmed porous microcapsules 21.

The microtube blanketing film 19 is formed of silicone and is 0.1 to 2 nm in pore side or mesh size. The film corresponds to organism diffusion preventing film, produced substance diffusion preventing film, and interfering substance preventing film. Since this microtube blanketing film 19 has the above-mentioned pore size or mesh size, it permits the permeation of glucose as the object of measurement but does not permit the permeation of the following: GOD genetically-modified bacteria 23, enzyme GOD produced by the GOD genetically-modified bacteria 23, and substances (various proteins and the like existing in the humor) which interfere with detection by the sensing element 15.

The polymer-filmed porous microcapsules 21 are formed of silicone and are 3 nm or so in pore size or mesh size, and correspond to organism diffusion preventing film. Since these polymer-filmed porous microcapsules 21 have the above-mentioned pore size or mesh size, they permit the permeation of enzyme GOD produced by the GOD genetically-modified bacteria 23 but do not permit the permeation of the GOD genetically-modified bacteria 23.

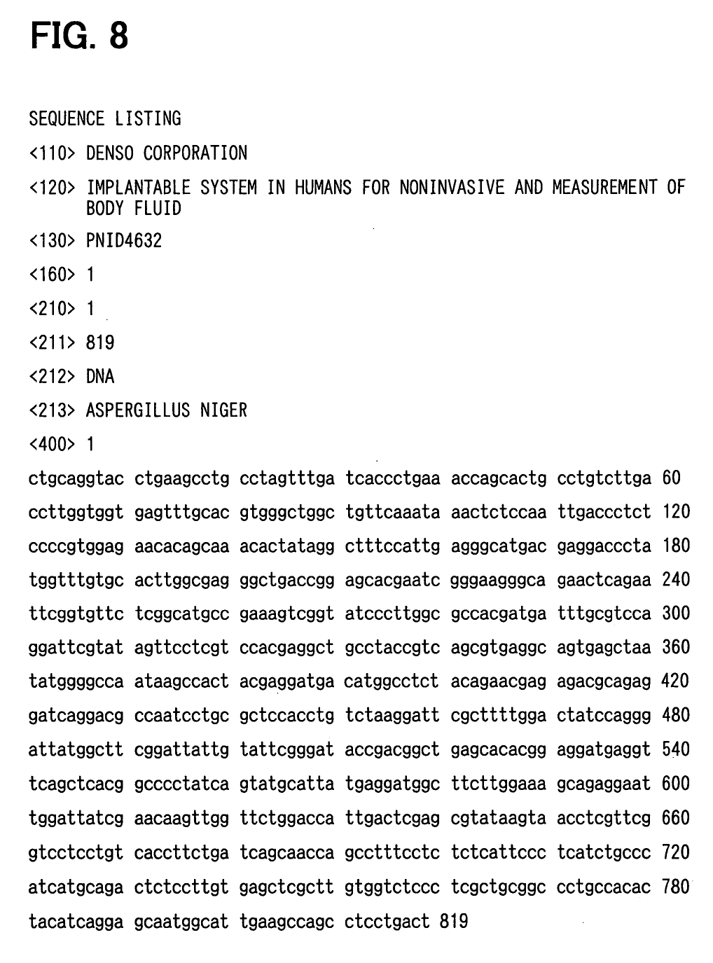

The GOD genetically-modified bacterium 23 is a microbe corresponding to the NSBI (National Center of Biotechnology Information)'s identification number of X56443. The X56443 is obtained by genetically modifying Escherichia coli as the host so as to produce GOD. A sequence listing described in FIG. 8 shows a base sequence of the X56443 gene.

The outer shell 9 is formed of porous silicone and is 0.1 to 2 nm in pore size or mesh size, and corresponds to organism diffusion preventing film, produced substance diffusion preventing film, interfering substance preventing film, and outer shell. Since the outer shell 9 has the above-mentioned pore size or mesh size, it permits the permeation of glucose in the humor but does not permit the permeation of the GOD genetically-modified bacteria 23, enzyme GOD produced by the GOD genetically-modified bacteria 23, and substances (various proteins and the like existing in the humor) which interfere with detection by the sensing element 15.

As illustrated in FIG. 1, the extra-corporeal sensing device 5 comprises a disk-shaped body portion 25 having a clockface, and a belt portion 27 for wearing the device around a wrist.

(b) Next, description will be given to a method by which the GOD genetically-modified bacteria 23 were obtained.

(i) First, mRNA was extracted from A.niger by the AGPC method. More specifically, the following procedure was taken: GTC (guanidine thiocyanate) was dissolved in distilled water, and then sodium acid citrate and sarcosyl (sodium N-lauroyl sarcosine) were added. The mixture was heated to 60 to 65° C. and stirred. 2-ME (2-mercaptoethanol) was added to obtain a denaturing solution. The denaturing solution was poured into a microtube, and cells or tissues were added to suspend the solution. Next, sodium acetate, equilibrium acid phenol, and CIA (chloroform/isoamyl alcohol) were added to the denaturing solution and mixed. When the mixture was left on ice for a while, it was separated into two layers: water layer and organic layer. The mixture was centrifugally separated for 20 minutes. The separated upper layer (water layer) was recovered into another tube, and isoproalcohol was added to the residue, which was in turn left standing at room temperature for 10 minutes. Thereafter, the mixture was centrifuged at 4° C. for 10 minutes, and the supernatant was discarded. RNA was precipitated and pelletized. The thus obtained RNA pellets were dissolved in DEPC treated water (diethylpyrocarbonate), and used as samples.

(ii) Next, the mRNA extracted in Step (i) above was taken as a template, and using reverse transcription enzyme, the synthesis reaction (reverse transcription reaction) of DNA was caused to occur. Using this DNA as a template, PCR was carried out (RT-PCR method). More specifically, the following procedure was taken:

cDNA Amplification Using RT-PCR Amplification of GOD Gene

A mixture of the RNA obtained in Step (i) above, oligo(dT)12-18, and DEPC treated water (RNA sample/primer) was incubated at 70° C. for 10 minutes. Thereafter, the mixture was transferred onto ice, and was left standing for one minute or longer. A PCR buffer, MgCl₂, dNTP mix, and DTT were added in sequence, and the mixture was incubated at 42° C. for five minutes. Reverse transcriptase (e.g SUPERSCRIPT II from BRL) was added thereto and mixed, and the mixture was incubated at 42° C. for 50 minutes. Thereafter, the mixture was further incubated at 70° C. for 15 minutes, and the reaction was stopped. The mixture was cooled on ice, and then centrifuged. The reaction liquid is collected on the bottom of the tube, and Rnase H was added thereto. What was obtained by incubating this at 37° C. for 20 minutes was used as a sample of PCR (PCR product).

Purification of Vector DNA

Next, the purification of DNA fragments was carried out from the PCR product using the GFX PCR DNA and Gel Band Purification Kit. This kit makes it possible to purify DNA fragments using GFX. A collection tube was set on a GFX column, and 500 μL of capture buffer was dropped into the column. 40 μL of the PCR product was dropped, and then pipetting was carried out. Centrifugal separation was carried out at 15,000 rpm at 4° C. for 30 seconds to attach DNA to GFX, and other impurities were excluded. Next, the GFX column is set in a new collection tube, and 500 μL of wash buffer was dropped. Thereafter, centrifugal separation was carried out at 15,000 rpm at 4° C. for 30 seconds.

Next, to elute chromosome DNA from GFX, the GFX column was set in a microtube, and 50 μL of sterilized water was dropped. After one-minute incubation, centrifugal separation was carried out at 15,000 rpm at 4° C. for one minute to obtain a DNA solution. Next, the DNA fragments were cut by BamHI and HindIII. 10 μL of the DNA fragments, 5 μL of 10×K buffer, 1 μL of BamHI, 1 μL of HindIII, and 33 μL of sterilized water were dropped, and the mixture was incubated at 37° C. for one hour. Then phenolic treatment was carried out to obtain insert DNA. The adjustment of vector was carried out by subjecting pQE vector to restriction enzyme treatment by BamHI and HindIII. 1 μg of pQE vector, 5 μL of 10×K buffer, 1 μL of BamHI, 1 μL of HindIII, and 41 μL of sterilized water were dropped into a microtube, and the mixture was incubated at 37° C. for one hour. Then phenolic treatment was carried out to obtain vector DNA.

Ligation Reaction

0.5 μg of pQE vector and 1.5 μg of insert DNA were added to a 500-μL microtube. Further, 2 μL of ligation buffer of tenfold concentration, 2.5 μL of 20-mg/mL BSA solution, and 1 μL (300 units) of T4 DNA Ligase were added, and finally sterilized water was added so that the total quantity would be 20 μL. The mixture was caused to undergo reaction at 16° C. for 1.5 hours.

Transformation

50 μL of pGAPZ α A, B, C (yeast from Invitrogen) and 5 μL of the ligation product were added to a microtube, and incubated in ice for 15 minutes. Thereafter, heat shock was applied at 42 ° C. for five minutes. After two-minute incubation in ice, shaking incubation was carried out in a SOC culture medium for one hour.

Confirmation of Gene Introduction

A culture medium to which 2.0% tryptone peptone, 0.5% yeast extract, 0.5% NaCl, and 1.5% agar were added was adjusted, and autoclaved at 121° C. for 15 minutes. After this culture medium was cooled to approximately 50° C., 0.1 mg/mL of ampicillin, 1 mM (0.286 mg/mL) of IPTG (Isopropyl-β-thiogalactoside), and 4 mg/mL of X-gal were added, and the mixture was dispensed into fertilized petri dishes in increments of 10 mL to create blue/white selection culture media. When IPTG is added to a culture medium, the expression of β-gal which is usually suppressed by repressor is relieved, and the induction of enzyme becomes prone to occur. As a result, a large quantity of a target product can be obtained.

As mentioned above, pGAPZ α A, B, C (yeast from Invitrogen) was used for the host. However, the present invention is not limited to this, and a wide variety of fungi and yeast which do not have pathogenicity to the human body and can sustain their lives at 25 to 37° C. under neutral pH conditions. (Examples of such fungi and yeast include Saccaromyces cerevisiae, repens, and oryzae.)

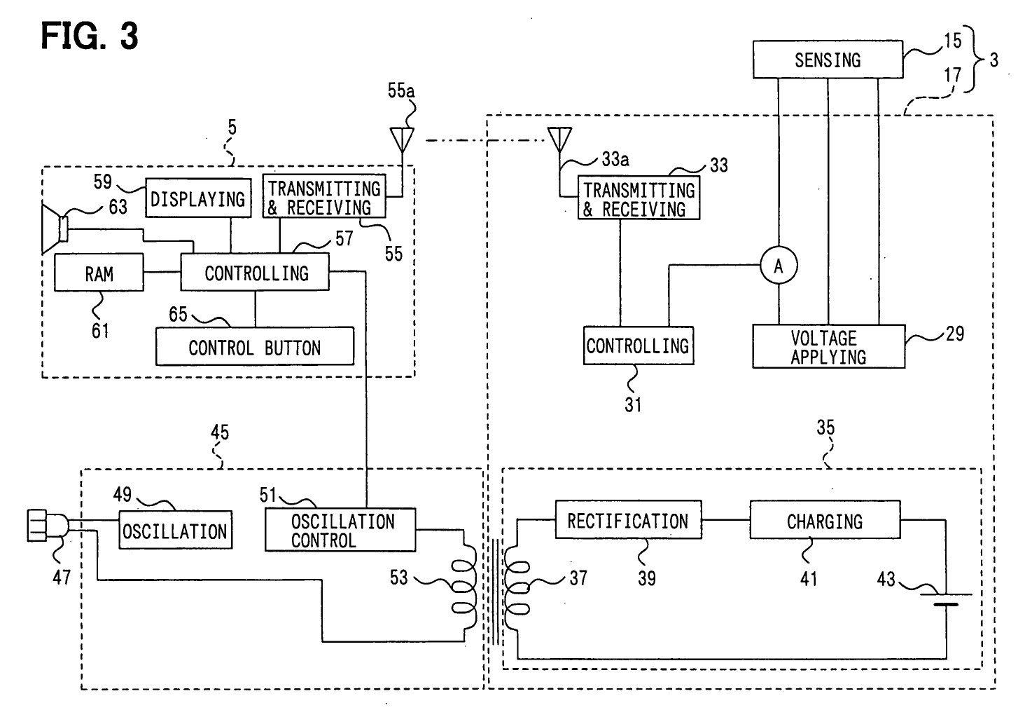

(c) Next, description will be given to the electrical constitution of the humor component detection device 3 and the extra-corporeal sensing device 5 with reference to FIG. 3.

The electronic device 17 of the humor component detection device 3 comprises a constant voltage applying portion 29, a controlling portion 31, and a transmitting and receiving portion 33. The constant voltage applying portion 29 applies constant voltage to the electrodes of the sensing element 15. The controlling portion 31 measures the value of current passed through the electrodes of the sensing element 15 with constant voltage applied thereto for a certain period of time (10 minutes or so), and transmits the obtained data to the transmitting and receiving portion 33. The transmitting and receiving portion (transmitting unit) 33 transmits the data to outside the human body through an antenna 33a in real time.

The electronic device 17 of the humor component detection device 3 is provided with a power supply portion 35 which functions as a power source for the constant voltage applying portion 29, controlling portion 31, and transmitting and receiving portion 33. The power supply portion 35 comprises a secondary coil 37, a rectification circuit 39, a charging circuit 41, and a secondary battery 43. The power supply portion 35 is capable of charging the device 3 using a charger 45, illustrated in FIG. 3, while the subject is sleeping or on like occasions. More specific description will be given. The charger 45 produces alternating-current voltage in a primary coil 53, using an AC power source 47, an oscillation circuit 49, and an oscillation control circuit 51. When this primary coil 53 is placed in proximity to the secondary coil 37 on the humor component detection device 3, electromotive force is produced in the secondary coil 37 by electromagnetic coupling. Based on this electromotive force, the secondary battery 43 is charged through the rectification circuit 39 and the charging circuit 41.

The extra-corporeal sensing device 5 comprises a transmitting and receiving portion (receiving unit) 55, a computing and controlling portion 57, a displaying portion 59, RAM 61, a speaker 63, and an external control button 65. The transmitting and receiving portion 55 receives data, transmitted from the humor component detection device 3, through an antenna 55a, and passes the data to the computing and controlling portion 57. The computing and controlling portion 57 carries out computation with the data, and displays the data on the displaying portion 59. At the same time, the computing and controlling portion 57 stores the data in the RAM (data recording unit) 61. The display style of the displaying portion 59 can be changed through the operation of the external control button 65. More specifically, past data stored in the RAM 61 can be read, and measurement data can be displayed in chronological order.

The computing and controlling portion (determining unit) 57 operates as follows: when the value of data is higher or lower than a preset reference value, it causes the displaying portion (notifying unit) 59 to display that. At the same time, the computing and controlling portion 57 causes the speaker (notifying unit) 63 to report the anomaly to alert the subject.

The extra-corporeal sensing device 5 is also capable of controlling the above-mentioned charger 45. More specific description will be given. With the charger 45 loaded into the extra-corporeal sensing device 5, the computing and controlling portion 57 of the extra-corporeal sensing device 5 controls the oscillation control circuit 51 of the charger 45.

(d) Next, description will be given to the action of the humor component detection device 3 and the humor component detection system 1 of the first embodiment during measurement with reference to FIG. 2.

As illustrated in FIG. 2, the humor component detection device 3 is hypodermically implanted (in the living body) using a publicly known syringe 67, and retained there. The place in which the humor component detection device 3 is retained may be inside the mouth, the back of an eyelid, or the like.

Glucose as the humor component to be measured permeates the outer shell 9 and the microtube blanketing film 19 and goes inside the microtube blanketing film 19. The pore size or mesh size of the outer shell 9 and the microtube blanketing film 19 is larger than glucose; therefore, glucose can permeate them.

The GOD genetically-modified bacteria 23 produce enzyme GOD. As mentioned above, enzyme GOD is smaller than the pore size or mesh size of the polymer-filmed porous microcapsules 21; therefore, enzyme GOD diffuses to outside the polymer-filmed porous microcapsules 21. However, the size of enzyme GOD is larger than the pore size or mesh size of the microtube blanketing film 19; therefore, enzyme GOD stays inside the microtube blanketing film 19.

As a result, glucose and enzyme GOD exist together inside the microtube blanketing film 19. As indicated by Formula (1), glucose undergoes chemical reaction in the presence of enzyme GOD inside the microtube blanketing film 19.
C₆H₁₂O₆+O₂→C₆H₁₀O₆+H₂O₂ Formula (1)

H₂O₂ produced according to Formula (1) undergoes chemical reaction at the working electrode (anode) as indicated by Formula (2).
H₂O₂→2H⁺+O₂+2e⁻ Formula (2)

H⁺ produced as the result of this reaction undergoes chemical reaction at the counter electrode (cathode) as indicated by Formula (3).
2H⁺+½O₂+2e⁻→H₂O Formula (3)

As the result of the occurrence of the chemical reactions expressed by Formula (2) and Formula (3), a current is passed between the anode and the cathode. As mentioned above, constant voltage is applied to between the anode and the cathode by the constant voltage applying portion 29. (Refer to FIG. 3.) Hence, the value of current depends on the quantity of glucose. Therefore, the quantity of glucose can be determined by measuring the value of current.

The electronic device 17 records the value of current as measurement data. It also converts the measurement data into radio signals and transmits the signals in real time. The extra-corporeal sensing device 5 receives radio signals transmitted from the humor component detection device 3, and stores them as measurement data.

(e) Next, description will be given to the effect brought about by the humor component detection device 3 and the humor component detection system 1 of the first embodiment.

(i) Since the humor component detection device 3 of the first embodiment is provided with the GOD genetically-modified bacteria 23, it can cause enzyme GOD to be continuously produced. For this reason, enzyme GOD required for measurement with the biosensor 6 is prevented from being devitalized, and measurement can be continuously carried out for a long time.

(ii) The humor component detection device 3 of the first embodiment measures glucose using the biosensor 6 based on electrochemical reaction. Therefore, it is capable of more accurate measurement than with methods using reflection spectrum.

(iii) Use of the humor component detection device 3 of the first embodiment obviates necessity for intracorporeally retaining a puncturing needle unlike conventional measuring methods, and the detection device 3 is excellent from the aspect of good hygiene.

(iv) The humor component detection device 3 of the first embodiment uses the genetically-modified bacteria 23 as the microbe for producing enzyme. Therefore, the detection device 3 is also capable of measurement related to diseases other than diabetes. More specific description will be given. Genetically modified bacteria can be caused to produce substances, such as enzyme, in addition to GOD, for use in measurement with the biosensor 6 by genetic engineering. Therefore, measurement related to any other disease can be carried out by causing the genetically modified bacteria to produce substances required for measuring measurement items corresponding to that disease.

(v) In the humor component detection device 3 of the first embodiment, the GOD genetically-modified bacteria 23 are covered with the microtube blanketing film 19 and the polymer-filmed porous microcapsules 21. The microtube blanketing film 19 and the polymer-filmed porous microcapsules 21 are smaller in pore size or mesh size than the GOD genetically-modified bacteria 23. Therefore, there is no possibility that the GOD genetically-modified bacteria 23 leak out of them.

(vi) In the humor component detection device 3 of the first embodiment, the tip 10a of the working electrode 10, the tip 11a of the counter electrode 11, and the tip 13a of the reference electrode 13 are covered with the microtube blanketing film 19. The pore size or mesh size of the microtube blanketing film 19 is smaller than substances (various proteins and the like existing in the humor) which interfere with detection by the sensing element 15. Therefore, interfering substances can be prevented from going inside the microtube blanketing film 19. For this reason, in the humor component detection device 3 of the first embodiment, the interfering substances are prevented from sticking to the electrodes of the sensing element 15, and accurate measurement can be constantly carried out.

The pore size or mesh size of the microtube blanketing film 19 is smaller than enzyme GOD. Therefore, the enzyme GOD produced by the GOD genetically-modified bacteria 23 stays inside the microtube blanketing film 19. For this reason, the concentration of enzyme GOD is sufficiently enhanced in proximity to the tip 10a of the working electrode 10, the tip 11a of the counter electrode 11, and the tip 13a of the reference electrode 13 existing inside the microtube blanketing film 19. As a result, accurate measurement can be carried out.

(vii) The humor component detection device 3 of the first embodiment is provided with the columnar outer shell 9 and houses the biosensor 6 and the microbe containing portion 7 therein. For this reason, the humor component detection device 3 can be smoothly hypodermically implanted using a syringe 67.

(xiii) In the humor component detection system 1 of the first embodiment, the humor component detection device 3 is provided with the transmitting and receiving portion 33, and the extra-corporeal sensing device 5 is provided with the transmitting and receiving portion 55. Thereby, the humor component detection system 1 is capable of wirelessly communicating measurement data in real time.

(ix) The humor component detection system 1 of the first embodiment is capable of determining whether received measurement data is abnormal or not by the computing and controlling portion 37 of the extra-corporeal sensing device 5. When measurement data is determined to be abnormal, that can be notified of by the displaying portion 59 and the speaker 63. Thus, the user can swiftly learn any anomaly in measurement data.

### Second Embodiment

With respect to configuration and action, a humor component detection system 1 of a second embodiment is basically the same as of the first embodiment, excepting a method for obtaining GOD genetically-modified bacteria 23. Hereafter, specific description will be given mainly to this difference.

In the second embodiment, the GOD genetically-modified bacteria 23 are obtained as follows:

(i) Extraction of Chromosome DNA from E. Coli

E. coli JM105 was shaking-cultivated in liquid until the absorbance OD₆₀₀ became 1 at 37° C., and the absorbance of the culture solution was adjusted until OD₆₀₀ became 5.0. This culture medium was dispensed into microtubes in increments of 1 mL, and centrifugal separation was carried out at 15,000 rpm at 25° C. for 30 seconds. Then, supernatant was completely removed. 40 μL of protein K buffer is added thereto, and the mixture was immediately and completely suspended and mixed by a vortex mixer. 10 μL of proteinase K is dropped into the mixture, and incubation was carried out at 55° C. for 15 minutes to break the cell walls. Further, 5 μL of RNase was added, and incubation was carried out at 25° C. for 10 minutes to decompose RNA. Next, 500 μL of extraction solution was added, and incubation was carried out at 25° C. for 10 minutes to completely break fungal forms. Fungal form extraction liquid was transferred to GFX column set in a collection tube, and centrifugal separation was carried out at 7,500 rpm at 25° C. for 15 minutes to attach DNA to GFX, and other impurities were excluded.

Next, the GFX column was set in a new collection tube, and 500 μL of extraction solution was added again to completely dissolve impurities. Centrifugal separation was carried out at 7,500 rpm at 25° C. for one minute to completely remove the waste liquid collected in the collection tube. 500 μL of wash solution was added to this GFX column, and centrifugal separation was carried out at 15,000 rpm at 25° C. for three minutes. The chromosome DNA attaching to GFX was washed. Centrifugal separation was carried out at 15,000 rpm at 25° C. for one minute, and GFX was completely dried to remove the wash solution.

Next, to elute chromosome DNA from GFX, the GFX column was set in a microtube, and 100 μL of TE buffer was added. After one-minute incubation at 25° C., centrifugal separation was carried out at 7,500 rpm at 25° C. for one minute to obtain a DNA solution.

(ii) Acquisition of Insert DNA and Vector DNA Using PCR Amplification of GOD Gene

1 μg of chromosome DNA, 5 μL of 10×Ex Taq™ buffer, 4 μL of dNTP mixture, 0.5 ρL of 5′ primer, 0.5 μL of 3′ primer, and 0.5 μL of TaKaRa Ex Taq were added to the microtube. The temperature condition was as follows: incubation was carried out at 98° C. for five minutes, and then it was repeated 30 times at 98° C. for 10 seconds, at 65° C. for 30 seconds, and at 72° C. for 90 seconds.

Next, the purification of DNA fragments was carried out from the PCR product using the GFX PCR DNA and Gel Band Purification Kit. This kit makes it possible to purify DNA fragments using GFX. A collection tube was set on a GFX column, and 500 μL of capture buffer was dropped into the column. 40 μL of the PCR product was dropped, and then pipetting was carried out. Centrifugal separation was carried out at 15,000 rpm at 4° C. for 30 seconds to attach DNA to GFX, and other impurities were excluded. Next, the GFX column is set in a new collection tube, and 500 μL of wash buffer was dropped. Thereafter, centrifugal separation was carried out at 15,000 rpm at 4° C. for 30 seconds.

Next, to elute chromosome DNA from GFX, the GFX column was set in a microtube, and 50 μL of sterilized water was dropped. After one-minute incubation, centrifugal separation was carried out at 15,000 rpm at 4° C. for one minute to obtain a DNA solution. Next, the DNA fragments were cut by BamHI and HindIII. 10 μL of the DNA fragments, 5 μL of 10×K buffer, 1 μL of BamHI, 1 μL of HindIII, and 33 μL of sterilized water were dropped into the microtube, and the mixture was incubated at 37° C. for one hour. Then phenolic treatment was carried out to obtain insert DNA. The adjustment of vector was carried out by subjecting pQE vector to restriction enzyme treatment by BamHI and HindIII. 1 μg of pQE vector, 5 μL of 10×K buffer, 1 μL of BamHI, 1 μL of HindIII, and 41 μL of sterilized water were dropped into the microtube, and the mixture was incubated at 37° C. for one hour. Then phenolic treatment was carried out to obtain vector DNA.

(iii) Ligation Reaction

0.5 μg of pQE vector and 1.5 μg of insert DNA were added to a 500-μL microtube. Further, 2 μL of ligation buffer of tenfold concentration, 2.5 μL of 20-mg/mL BSA solution, and 1 μL (300 units) of T4 DNA Ligase were added, and finally sterilized water was added so that the total quantity would be 20 μL. The mixture was caused to undergo reaction at 16° C. for 1.5 hours.

(iv) Transformation

50 μL of competent cells and 5 μL of the ligation product were added to the microtube, and incubated in ice for 15 minutes. Thereafter, heat shock was applied at 42° C. for five minutes. After two-minute incubation in ice, shaking incubation was carried out in a SOC culture medium for one hour.

(v) Confirmation of Gene Introduction

A culture medium to which 2.0% tryptone peptone, 0.5% yeast extract, 0.5% NaCl, and 1.5% agar were added was adjusted, and autoclaved at 121° C. for 15 minutes. After this culture medium was cooled to approximately 50° C., 0.1 mg/mL of ampicillin, 1 mM (0.286 mg/mL) of IPTG (Isopropyl-β-thiogalactoside), and 4 mg/mL of X-gal were added, and the mixture was dispensed into fertilized petri dishes in increments of 10 mL to create blue/white selection culture media. When IPTG is added to a culture medium, the expression of β-gal which is usually suppressed by repressor is relieved, and the induction of enzyme becomes prone to occur. As a result, a large quantity of a target product can be obtained. 100 μL of genetically introduced E. coli JM109 is implanted into this culture medium, which is cultivated overnight at 37° to obtain a target bacterium.

### Third Embodiment

With respect to configuration and action, a humor component detection system 1 of a third embodiment is basically the same as of the first embodiment. However, they are partially different from each other in the configuration of the humor component detection device 3. Hereafter, specific description will be given mainly to this difference.

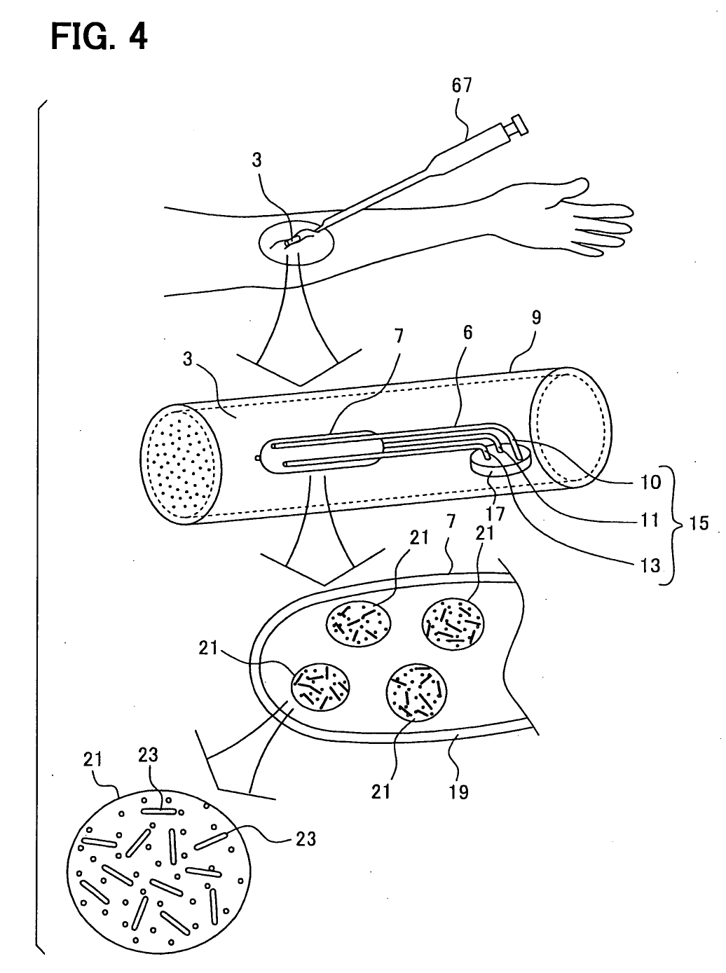

In the humor component detection device 3 of the third embodiment, as illustrated in FIG. 4, the microbe containing portion 7 is positioned between the three electrodes 10, 11, and 13 of the sensing element 15. Therefore, in the third embodiment, the electrodes 10, 11, and 13 are positioned outside the microbe containing portion 7.

As in the first embodiment, the microbe containing portion 7 has the pouched microtube blanketing film 19, a plurality of the acinous polymer-filmed porous microcapsules 21 contained inside the microtube blanketing film 19, and the GOD genetically-modified bacteria 23 contained in the polymer-filmed porous microcapsules 21.

In the third embodiment, the pore size or mesh size of the microtube blanketing film 19 is 0.1 to 1 μm. It does not permit the permeation of the GOD genetically-modified bacteria 23 but permits enzyme GOD produced by the GOD genetically-modified bacteria 23 to permeate to the outside. At the electrodes 10, 11, and 13 of the sensing element 15, the same electrochemical reaction as in the first embodiment occurs with respect to glucose in the presence of enzyme GOD which has permeated across the microtube blanketing film 19. Therefore, the sensing element 15 can detect the value of current corresponding to the quantity of glucose.

The humor component detection device 3 and the humor component detection system 1 of the third embodiment bring about the same effects as the first embodiment. In the third embodiment, however, substances (various proteins and the like existing in the humor) which interfere with measurement are prevented from sticking to the electrodes of the sensing element 15 by the outer shell 9, not by the microtube blanketing film 19. In the third embodiment, a role to prevent the diffusion of enzyme GOD and keep the concentration of enzyme GOD at a certain value or higher at the sensing element 15 is fulfilled by the outer shell 9, not by the microtube blanketing film 19.

### Fourth Embodiment

With respect to configuration and action, a humor component detection system 1 of a fourth embodiment is basically the same as of the first embodiment, excepting the electrodes 10, 11, and 13 of the sensing element 15 in the humor component detection device 3. In the fourth embodiment, the surfaces of the electrodes 10, 11, and 13 are chemically modified with silicone which is one of living body functional polymers. For this reason, in the fourth embodiment, the interfering substances in the humor can be more effectively prevented from sticking to the electrodes, and accurate measurement can be carried out for a long time.

### Fifth Embodiment

With respect to configuration and action, a humor component detection system 1 of a fifth embodiment is basically the same as of the first embodiment. However, they are partially different from each other in the configuration of the humor component detection device 3. Hereafter, specific description will be given mainly to this difference.

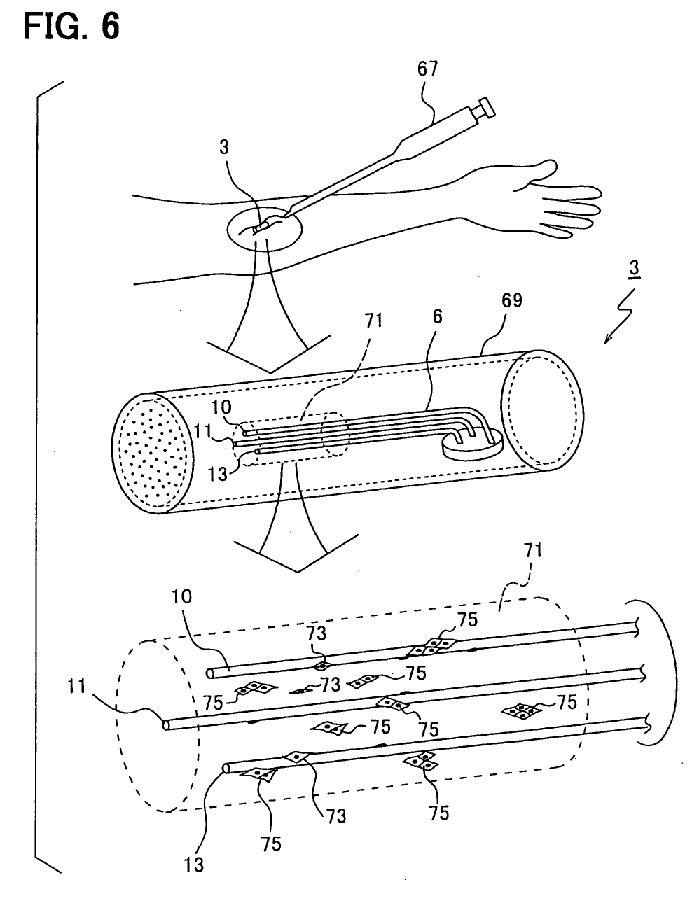

As illustrated in FIG. 6, the humor component detection device 3 is made from porous silicone, and comprises a body portion 69 having a columnar shape and the same biosensor 6 as in the first embodiment, embedded therein.

The body portion 69 is filled with porous silicone, excepting a columnar air space portion 71 positioned therein. With respect to the biosensor 6, the tips of the three electrodes, working electrode 10, counter electrode 11, and reference electrode 13, are positioned in the air space portion 71. The other portions of the biosensor 6 are embedded in the porous silicone in the body portion 69.

The air space portion 71 contains physiological saline as well as a mixture of modified cells 73 having genetic information for autoreproduction and tissues 75 composed of a plurality of the modified cells 73. These modified cells 73 and tissues 75 produce enzyme GOD. Using this enzyme GOD, the biosensor 6 measures the quantity of glucose as in the first embodiment.

In the fifth embodiment, the body portion 69 is made from porous silicone with a pore size or mesh size of 0.1 to 2 nm, and it corresponds to organism diffusion preventing film, produced substance diffusion preventing film, and interfering substance preventing film. More specific description will be given. Since the body portion 69 has the above-mentioned pore size or mesh size, it permits the permeation of glucose as the object of measurement, but does not permit the diffusion of the modified cells 73, tissues 75, or enzyme GOD produced by the modified cells 73 or the tissues 75 to outside the body portion 69. Further, the body portion 69 does not permit the permeation of substances (various proteins and the like existing in the humor) which interfere with detection by the biosensor 6 into the air space portion 71.

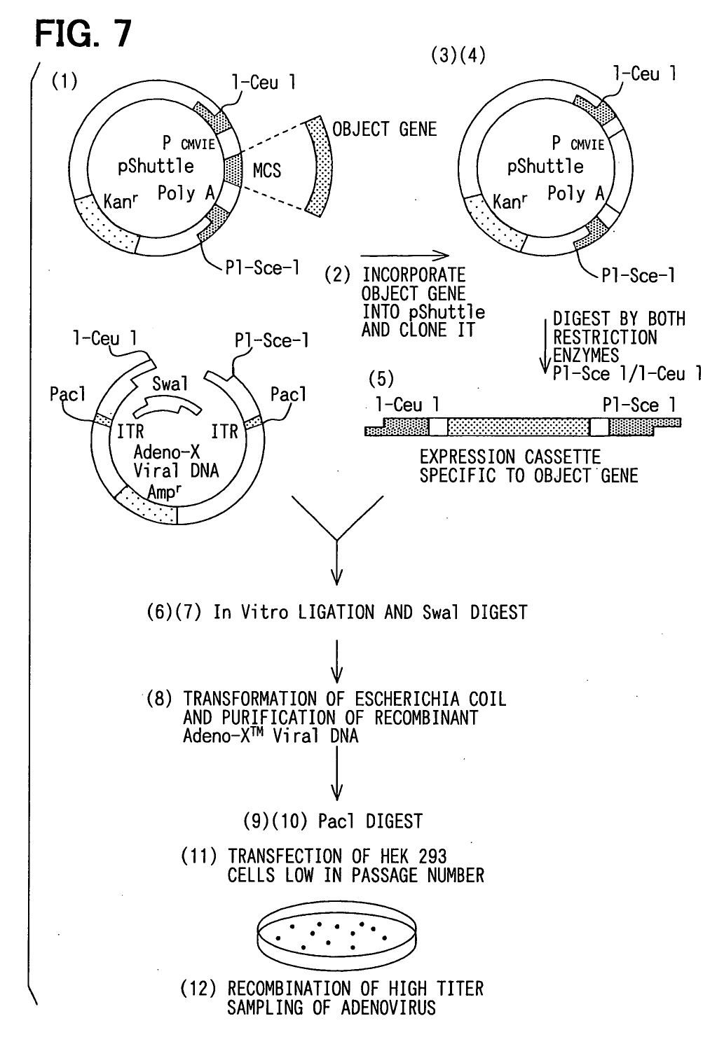

Next, the modified cells 73 were created in accordance with the procedure described under Items (1) to (13) below. This method for creation will be described with reference to FIG. 7. The method uses a technique of infecting target cells using the Adeno-XExpression System (registered trademark) kit from Clontech. pShuttle Vector is used to construct an expression cassette specific to the genes of mammals in which the I-Ceu and PI-Sce-I restriction enzyme portions are positioned at both ends.

(1) Adjustment of Plasmid

A recombinant pShuttle vector specific to genes is constructed by a standard molecular biological technique.

(2) Cloning of Object Gene

Object genes are incorporated into the pShuttle and cloned.

(3) Transformation of Escherichia Coli

Vectors digested by restriction enzyme and gene fragments are ligated, and the resulting product is transformed into Escherichia coli.

(4) Purification of Plasmid DNA

Restriction enzyme sites are analyzed to identify object recombinant plasmid. The orientation and binding site of fragments inserted by sequencing are confirmed. When identified, a large quantity is prepared, and all plasmids for transfection are isolated.

(5) Excision of Expression Cassette from pShuttle

Using restriction enzymes PI-Sce I and I-Ceu I, expression cassettes are excided from the pShuttle plasmid DNA.

(6) Ligation of Expression Cassette into Adeno-X Viral DNA

The excided expression cassettes are incorporated into Adeno-X Viral DNA by in vitro ligation reaction.

(7) Cutting of Ligation Product

The ligation product is cut by SwaI.

(8) Transformation of Escherichia Coli

Using a recombination deficient host strain, such as DH5α, for general purposes, chemically or electrically competent Escherichia coli is transformed by a standard molecular biological technique.

(9) Purification of SwaI Digest of Recombinant Adenovirus DNA Containing Object Gene

Recombinants are identified by an analysis of restriction enzyme sites.

(10) Digestion of Recombinant Adenovirus DNA by Pac I

The recombinant adenovirus is digested by restriction enzyme Pac I.

(11) Transfection of Pac I-digested Recombinant Adenovirus DNA into HEK293 cell

Using HEK293 cells, the above-mentioned SwaI digest of adenovirus DNA is transfected to package and proliferate the vectors arising from adenovirus.

The HEK293 cells are cultivated beforehand and the stock is maintained. To store the cells for a long time, they are refrigerated.

(12) Sampling of Recombinant Adenovirus

Adenovirus is sampled, and virus titer measurement is carried out to determine viral activity.

(13) Infection of Target Cell

The target cells are infected with adenovirus to create the modified cells 73.

Next, the tissues 75 were obtained from the modified cells 73 in accordance with the procedure described under Items (1) to (6) below. The following method is an incubator technique and the OptiCell (registered trademark) from BioCrystal was used for the instrument for this purpose.

(1) The access port of the Opticell is thoroughly wiped with cotton with alcohol.

(2) The cap of a culture medium bottle is sterilized with cotton with alcohol, and the culture medium is sucked by a syringe.

(3) A chip is inserted into the access port of the Opticell, and the culture medium and the modified cells 73 are injected.

(4) The Opticell is turned upside down, and is caused to suck 10 ml of air.

(5) The access port of the Opticell is wiped with cotton with alcohol, and cultivation is carried out in an incubator.

(6) Air is injected into the Opticell, and then the culture medium is sucked. Using forceps or the like, it is cut off from one positive adherence face along the frame to obtain the cultivated cells 75.

The humor component detection device 3 and the humor component detection system 1 of the fifth embodiment bring about the same effects as the first embodiment.

(Others)

Embodiments of the present invention are not limited to those mentioned above at all, and needless to add, the present invention can be modified in various manners to the extent that the sprit and scope of the present invention are not departed from.

Some examples will be taken. In the above-mentioned first embodiment to fifth embodiment, the humor component detection device 3 is capable of transmitting the state of the power supply portion 35 together with measurement data using the transmitting and receiving portion 33. In this case, the extra-corporeal sensing device 5 receives the state of power supply together with the measurement data, and is capable of monitoring the completion of charging of the power supply portion 35 and reduction in the capacity of the secondary battery 43.

In the above-mentioned first embodiment to fourth embodiment, the polymer-filmed porous microcapsules 21 may be eliminated. In this case, the GOD genetically-modified bacteria 23 dispersedly exist inside the microtube blanketing film 19. The pore size or mesh size of the microtube blanketing film 19 is smaller than the GOD genetically-modified bacteria 23; therefore, the GOD genetically-modified bacteria 23 are prevented from leaking to outside the microtube blanketing film 19.

In the above-mentioned first embodiment to fourth embodiment, the microbe which produces GOD is not limited to X56443, and may be any other bacterium. Possible microbes include those corresponding to the following NSBI identification numbers: CB360053, NT_{—}039553, BC012279, NM_{—}013929, AK017570, AK010562, AB095542, XM_{—}122470, AF483594, AF483582, BB001275, AF220557, AF220556, AF220555, AF214704, and the like.

In the above-mentioned first embodiment to fifth embodiment, the humor component detection system is constituted as a system for detecting the condition of diabetes. It may be constituted as a system for detecting the condition of lifestyle-related diseases, hyperlipemia, or pancreatitis or as a metabolism monitor.

When the humor component detection system is constituted as a system for detecting the condition of hyperlipemia, the humor component to be measured is cholesterol. In the first embodiment to the fourth embodiment in this case, the microbe containing portion 7 contains genetically modified bacteria which produce cholesterol esterase and cholesterol oxidase as enzymes. In the fourth embodiment, the air space portion 71 contains the modified cells 73 and the tissues 75 which produce cholesterol esterase and cholesterol oxidase as enzymes. At this time, the humor component detection system 1 is capable of detecting currents involved in the electrochemical reaction of cholesterol in the presence of cholesterol esterase and cholesterol oxidase. The transducer in this reaction is H₂O₂.

When the humor component detection system is constituted as a system for detecting the condition of pancreatitis, the humor component to be measured is α-amylase. In the first embodiment to the fourth embodiment in this case, the microbe containing portion 7 contains genetically modified bacteria which produce α-amylase, α-glucosidase, and glucose oxidase as enzymes. In the fifth embodiment, the air space portion 71 contains the modified cells 73 and the tissues 75 which produce α-amylase, α-glucosidase, and glucose oxidase as enzymes. At this time, the electrochemical reaction illustrated in FIG. 5 occurs at the sensing element 15 of the humor component detection device 3 in the presece of α-amylase, α-glucosidase, and glucose oxidase. The speed of this electrochemical reaction, that is, the value of current detected depends on the quantity of α-amylase. Therefore, the quantity of α-amylase can be determined by measuring the value of current. The transducer in the reaction illustrated in FIG. 5 is H₂O₂.

When the humor component detection system is constituted as a metabolism monitor, the humor component to be measured is lactic acid. In the first embodiment to the fourth embodiment in this case, the microbe containing portion 7 contains genetically modified bacteria which produce lactate oxidase as an enzyme. In the fifth embodiment, the air space portion 71 contains the modified cells 73 and the tissues 75 which produce lactate oxidase as an enzyme. At this time, the humor component detection system 1 is capable of detecting currents involved in the electrochemical reaction of lactic acid in the presence of lactate oxidase. The transducer in this reaction is H₂O₂.

It will be obvious to those skilled in the art that various changes may be made in the above-described embodiments of the present invention. However, the scope of the present invention should be determined by the following claims.

1. A humor component detection device retained in a living body, the humor component detection device comprising:
a biosensor which is capable of measuring a humor component; and
an organism which produces a substance used in the biosensor.

2. The humor component detection device according to claim 1,
wherein the organism includes microbes.

3. The humor component detection device according to claim 2,
wherein the microbes include transgenics bacteria.

4. The humor component detection device according to claim 1,
wherein the organism is any of (A) to (C) below:
(A) cells having genetic information for autoreproduction;
(B) tissues composed of the cells; and
(C) a mixture of the cells and the tissues.

5. The humor component detection device according to claim 4,
wherein the cells are of human origin.

6. The humor component detection device according to claim 1,
wherein the organism is covered with an organism diffusion preventing film which does not permit permeation of the organism.

7. The humor component detection device according to claim 1,
wherein the biosensor includes a detecting element, which performs detection of the humor component, and is covered with an interfering substance preventing film, and
wherein the interfering substance preventing film limits permeation of interfering substances against the detection and allows permeation of the humor component.

8. The humor component detection device according to claim 1,
wherein the biosensor includes a detecting element, which performs detection of the humor component, and is covered with a produced substance diffusion preventing film,
wherein the produced substance diffusion preventing film limits permeation of substances produced by the organism and allows permeation of the humor component, and
wherein the organism is contained inside the produced substance diffusion preventing film.

9. The humor component detection device according to claim 1, further comprising:
an outer shell which houses the biosensor and the organism and permits permeation of the humor component.

10. The humor component detection device according to claim 1,
wherein the biosensor includes, as a detecting element for detecting the humor component, electrodes which is able to detect electrical signals produced when the humor component undergoes electrochemical reaction.

11. The humor component detection device according to claim 10,
wherein the electrodes are chemically modified with living body functional polymer.

12. The humor component detection device according to claim 1, further comprising:
a transmitting unit which transmits data obtained as a result of detection by the biosensor, to an outside.

13. The humor component detection device according to claim 12,
wherein the transmitting unit wirelessly communicates the data in real time.

14. A humor component detection system comprising:
the humor component detection device that is retained in a living body and includes
a biosensor which is capable of measuring a humor component,
an organism which produces a substance used in the biosensor, and
a transmitting unit which transmits data obtained as a result of detection by the biosensor, to an outside; and
an extra-corporeal monitoring device provided with a receiving unit which is capable of receiving the data transmitted from the transmitting unit.

15. The humor component detection system according to claim 14,
wherein the extra-corporeal monitoring device has a data recording unit which stores the data received together with time data.

16. The humor component detection system according to claim 14,
wherein the extra-corporeal monitoring device includes:
a determining unit which determines whether the data received is abnormal or not; and
a notifying unit which, when the data is determined to be abnormal, notifies of an anomaly.
